(19) United States
(12) Patent Application Publication
(10) Pub. No.: US 2022/0068159 A1
(43) Pub. Date: Mar. 3, 2022

(54) SYSTEM AND METHOD OF PROVIDING SELF-HELP THROUGH DAILY TASKS

(71) Applicant: Adam Rich, Oakland, CA (US)

(72) Inventor: Adam Rich, Oakland, CA (US)

(21) Appl. No.: 17/004,743

(22) Filed: Aug. 27, 2020

(51) Int. Cl. G09B 19/00; G16H 20/70; G06Q 10/10; G06F 16/25

(52) U.S. Cl. CPC G09B 19/003; G16H 20/70; H04L 67/12; G06F 16/254; G06Q 10/1097

(57) ABSTRACT

A method includes receiving from an administrator device a set of actions for a user to carry out. The method also includes receiving from the administrator device subtasks of the actions. Further, the method includes storing the actions and subtasks into a database and receiving from the database, assessment factors, mood factors, and actions and tasks completed related to a user. Further still, the method includes sending to a user device, associated with the user, a subtask to be completed.

### TECHNICAL FIELD

The present disclosure relates to computer software for promoting self-help or life-enrichment, through use of daily tasks.

### BACKGROUND

Many people today are interested in changing their pattern of behavior in order to improve their lives, their mood, their health, or their well-being. To accomplish changes in behavior people may use self-help books, counselors, advisers, and programmable devices to aid in their progress. Many of these procedures require substantial amounts of time in order to change patterns of behavior or to make life improvements. Therefore, there is a need for improved systems and methods of promoting self-help or life-enrichment.

### SUMMARY

Various disclosed embodiments systems and methods for promoting self-help or life-enrichment, through use of simple and short daily tasks.

In some embodiments, a non-transitory computer readable medium having stored thereon software instructions that when executed by a processor, cause the processor to execute a method. The method includes receiving from an administrator device a set of actions for a user to carry out. The method also includes receiving from the administrator device subtasks of the actions. Further, the method includes storing the actions and subtasks into a database and receiving from the database, assessment factors, mood factors, and actions and tasks completed related to a user. Further still, the method includes sending to a user device, associated with the user, a subtask to be completed.

In other embodiments, a method includes starting a software application on a user device and logging in to a user account. The method also includes providing user check-in information to the software application. Further, the method includes sending the check-in information to a server in communication with a database and retrieving a daily task from the database. Further still, the method includes presenting the daily task to the user.

In yet other embodiments, a method includes content creation for a self-help software application. The method includes extracting from a reference material at least one of exercises, contentions, and wisdom. The method also includes converting the at least one of exercises, contentions, and wisdom into actions and associating a quote from the reference material with the at least one of the at least one of exercises, contentions, and wisdom. Further still, the method includes breaking the actions down into one or more steps that are day-by-day tasks for completing an action and explaining the actions as instructions.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Illustrative embodiments are illustrated in referenced figures of the drawings. It is intended that the embodiments and figures disclosed herein are to be considered illustrative rather than restrictive.

FIG. 1 is an illustrative flow diagram for the software in accordance with the invention.

FIG. 2 is an illustrative embodiment of a set of method steps in accordance with an invention.

FIG. 3 is an illustrative embodiment of a computing platform which can be configured for running software.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

In accordance with illustrative embodiments, a content platform may be designed to propel a greater sense of well-being through daily micro-actions. The platform may use inspirational self-help, advice, and other directive content, and break it into 10-minute exercises optimized to fit into the liminal moments in a busy day. In some embodiments the exercises may be of varied lengths not limited to 10-minutes. The exercises are best kept to a length where people will be engaged and find time to carry out the exercises on a regular basis.

In accordance with an illustrative embodiment, latent value in existing directive content may be extracted. Because of the power and flexibility of the digital product, there are many applications beyond adapting books, for example adapting video and audio, etc. In some embodiments, content is extracted from authors or performers who build loyal followings, however the subject matter disclosed may be adapted from any content, not just limited to well-known authors or performers.

In accordance with an illustrative embodiment, some certain attributes typically make for a good experience, e.g.:
1. Sound logic—Because the platform delivers daily wisdom couched in the research of positive psychology from expert partners such as Harvard's Human Flourishing Program, if works of dubious scientific credibility are used, before long the structure of the platform will reveal bad curation.
2. Advise—The book must go as far as to make specific recommendations. A collection of anecdotes won't supply sufficient fodder to bring the author's ideas into daily practice.
3. Audience—There must be a large and engaged existing audience for the author and their work. Check their social media footprint, inquire about newsletter distribution, etc.

There have been identified elements of positive psychological theory that serve as a framework for operationalizing an author's work. At its simplest, it is about a set of reflective activities that prompt feelings of well-being through reflection, perspective, and gratitude. And in presenting an audience with these small, achievable actions, a sense of accomplishment may be provided that compounds each day.

Examples of these actions, tasks, or subtasks are:

(i) journaling: unbroken time teasing out one's feeling in writing

(ii) writing a list or brainstorming: uncritical list-making intended to get as many ideas out as possible

(iii) communicating with someone: connecting with someone via whatever method feels most comfortable

(iv) writing a letter. This does not require it be sent, but rather is about imposing the social and logical framework of sharing these ideas with another person

(v) imagining spending time conjuring an outcome can help you visualize your success

(vi) group discussion: sharing thoughts with others and welcoming in new perspectives

(ix) joining a community: similar to a group discussion, but this has a longitudinal component intended to confer a sense of belonging and investment.

Typical users of various embodiments are busy, distracted, oversubscribed, and optimistic. They may have little time each day that they can really devote to a new thing. Also, their comfort zone may also be limited. When it comes to prompting daily actions, the whole philosophy is that tiny changes in the scope of an ask, or how familiar it feels are the difference between someone developing a healthy habit, and someone abandoning the product. Ask and comfort are the two qualities to dial in with the greatest sensitivity. In various embodiments, users are asked to identify a time in their day when they usually have, for example, 10 underutilized minutes. This way, they've indicated likely moments for the platform to present a modest ask.

Generating actions, tasks, or subtasks is key to providing a valuable experience for the user. Various embodiments include a method of content creation, including creating actions, tasks, or subtasks, for the self-help software application. These methods may include some or more of the following steps:
1) Extracting from a reference material at least one of exercises, contentions, and wisdom or other content.
2) Converting the at least one of exercises, contentions, and wisdom into actions.
3) Associating a quote from the reference material with the at least one of the at least one of exercises, contentions, and wisdom.
4) Breaking the actions down into one or more steps that are day-by-day tasks for completing an action.
5) Explaining the actions as instructions to the user.

These steps may be used to take information from reference books and convert it into sub tasks that may be performed by a user in, for example, less than 10 minutes.

Another illustrative process for generation of tasks may include the following steps.
A. Read the book and highlight exercises, contentions, wisdom, and other pearls that can be turned into actions
B. Review highlights and decide which action type best suits its intent.
C. Break the action into, for example, 10-minute steps. These can be of multiple different types, but each needs to be a doable level of ask and comfort, but still be significant-enough that our user will feel a burst of pride when they accomplish it.

Outputs that may be achieved by the platform include but are not limited to:
1. Quote from the book: this is the exercise or observation that inspires the action, taken verbatim from the work.
2. Instructions: explains the action itself within the context of the platform. The instructions connect the dots between the author's writing, and what the user will be doing each day
3. Steps: These are the day-by-day steps for completing the action

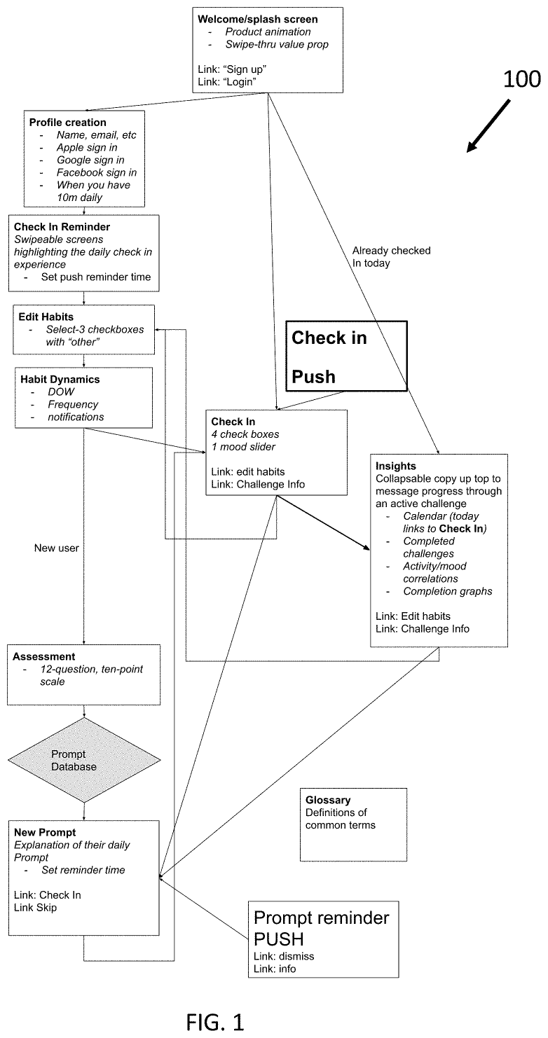

Referring now to FIG. 1, a flow diagram 100 for the platform's software is depicted. A welcome/splash screen is provided for a user login. In a first use, the user is provided with a Profile creation screen. A check-in reminder is provided where a user can set a time for a daily reminder notification. An Edit Habits screen may be accessed where a user is prompted to select from a list of habits that they wish to work on. A habit dynamics screen is accessed in which day of week, frequency, and notifications may be set. If the user is a new user, they are prompted to take an assessment questionnaire, which may be but is not limited to a 12-question questionnaire where the scale for each question may be a ten-point scale. Answers to the assessment are provided to the prompt database. The prompt database stores the different prompts, tasks, or sub-tasks and they may be selected for the user based on a number of criteria including but not limited to assessment factors, mood factors, and actions and tasks completed related to a user. The prompt database then provides a new prompt or daily task/subtask to a user. The user may then either complete the task or skip it. If skipped, the user may later be reminded to complete the prompt by a prompt reminder push which may be either accepted or dismissed. If the user is not a new user, the user may be asked to check in with a few questions and a mood slider to provide the user's mood. A user may opt to look at various Insights such as but not limited to Calendar with indications of days checked in, completed challenges, activity/mood correlations, completion graphs, and any other information.

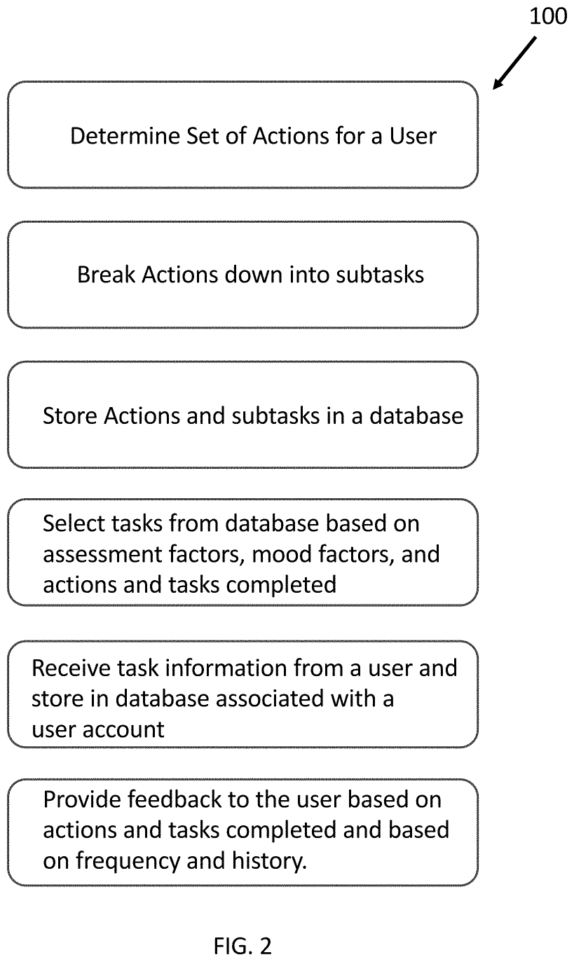

Referring now to FIG. 2, a set of steps 200 is depicted. Steps 200 include determining a set of actions for a user. The actions may be broken down into subtasks. These Actions and Subtasks are stored in a database. Based on various factors, such as but not limited to assessment factors, mood factors, and actions and tasks completed, a task is selected from the database. Task information may be received from the user and stored in the database associated with a user account. Feedback may be provided to the user based on actions and tasks completed and based on frequency and history.

As small ten-minute tasks are carried out by the user, the user benefits by improving on behavioral health, mood, skills, etc.

One challenge for providing value to a user of the software may be to consistently deliver users content (Challenges) that is both relevant and engaging. Relevance may be achieved by aligning the Challenge with the self-reported results of the assessment. Engagement may be predicted by making sure the Challenge is fresh, high-quality, and aligns with the kinds of content the user has historically engaged with.

In order to support this algorithm, the content database may include a schema which includes several parameters which are given here by way of example and are not thereby limiting:
1. Domain: as laid out by Harvard's Human Flourishing Program, well-being is comprised of six domains: Happiness and Life Satisfaction, Mental and Physical Health, Meaning and Purpose, Character and Virtue, Close Social Relationships, and Financial and Material Stability. Every piece of content will be tagged with which of these domains it addresses.
2. Intervention Type: HFP's research establishes a basic set of intervention types that, in combination with positive psychological direction, result in increased happiness, satisfaction, and a reduction in depression. These include Journaling, Brainstorming, Articulation, Visualization, Letter Writing, Conversation, and Group Discussion.
3. Engagement: this is a metric driven by broad engagement with the Flourish platform. It is described by how many times a piece of content has been viewed against how many times it has been marked DONE, and how many times it has been skipped
4. Has Been Done: simply whether a particular user has completed this piece of content in the past

On the completion of the Human Flourishing Index, the example algorithm may generate a custom Challenge list for each user. It may be created using the example following logic:
1. The first operation is to immediately remove any Challenges the user has already completed.
2. Based on the responses to the assessment, our content database is filtered down to the Challenges that address the three weakest domains.
3. From the user's historical engagement, it then removes all Challenges of any Intervention Type the user has consistently skipped and has never completed.
4. Then the list is ordered from most engaging (as determined by user engagement platform-wide) to least-engaging.
5. Finally, the list is shuffled to ensure no two challenges in a row address the same domain.

Using the example algorithm or similar adaptive algorithms may help to provide the user with an experience of greater value.

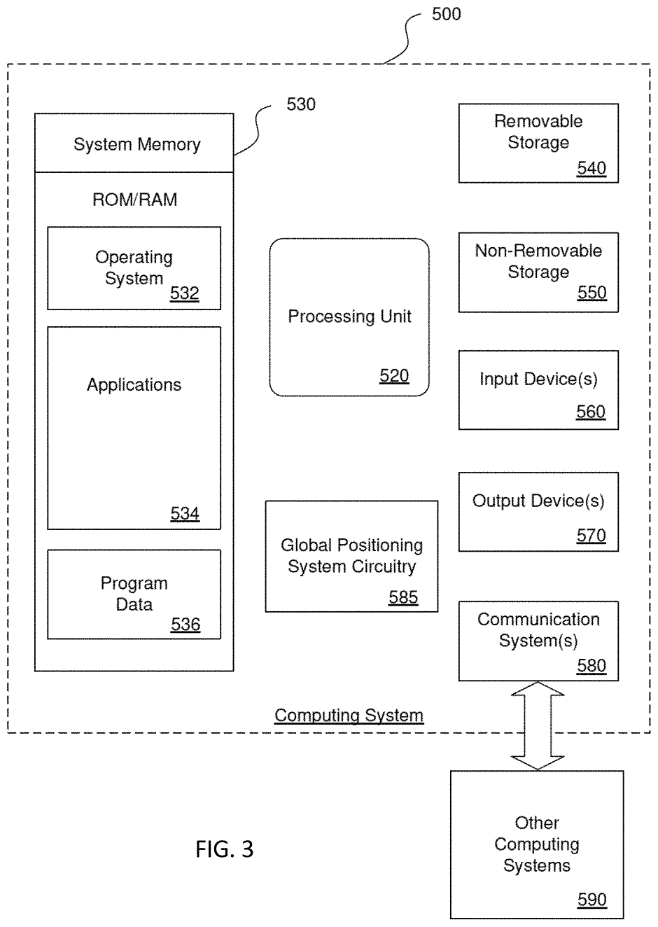

Referring now to FIG. 3 and given by way of example only and not of limitation, in various embodiments, the computing system 500 used for running the self-help software typically includes at least one processing unit 520 and a system memory 530. Depending on the exact configuration and type of computing device, the system memory 530 may be volatile memory, such as random-access memory (“RAM”), non-volatile memory, such as read-only memory (“ROM”), flash memory, and the like, or some combination of volatile memory and non-volatile memory. The system memory 530 typically maintains an operating system 532, one or more applications 534, and program data 536. The operating system 532 may include any number of operating systems executable on desktop or portable devices including, but not limited to, Linux, Microsoft Windows®, Apple OS®, or Android®, or a proprietary operating system.

The computing system 500 may also have additional features or functionality. For example, the computing system 500 may also include additional data storage devices (removable and/or non-removable) such as, for example, magnetic disks, optical disks, tape, or flash memory. Such additional storage is illustrated in FIG. 5 by removable storage 540 and non-removable storage 550. Computer storage media may include volatile and non-volatile, removable and non-removable media implemented in any method or technology for storage of information, such as computer-readable instructions, data structures, program modules or other data. The system memory 530, the removable storage 540, and the non-removable storage 550 are all examples of computer storage media. Available types of computer storage media include, but are not limited to, RAM, ROM, EEPROM, flash memory (in both removable and non-removable forms) or other memory technology, CD-ROM, digital versatile disks (DVD) or other optical storage, magnetic cassettes, magnetic disk storage or other magnetic storage devices, or any other medium which can be used to store the desired information and which can be accessed by the computing system 500. Any such computer storage media may be part of the computing system 500.

The computing system 500 may also have input device(s) 560 such as a keyboard, mouse, stylus, voice input device, touchscreen input device, etc. Output device(s) 570 such as a display, speakers, printer, short-range transceivers such as a Bluetooth transceiver, etc., may also be included. The computing system 500 also may include one or more communication systems 580 that allow the computing system 500 to communicate with other computing systems 590, for example, as the vehicle navigation control system 380 aboard the vehicle 300 (FIG. 3) communicates with the remote computing system 450 and vice versa. As previously mentioned, the communication system 580 may include systems for wired or wireless communications. Available forms of communication media typically carry computer-readable instructions, data structures, program modules or other data in a modulated data signal such as a carrier wave or other transport mechanism and includes any information delivery media. The term “modulated data signal” may include a signal that has one or more of its characteristics set or changed in such a manner as to encode information in the signal. By way of illustrative example only and not of limitation, communication media may include wired media such as a wired network or direct-wired connection, and wireless media such as acoustic, radio frequency (RF), infrared and other wireless media. The term computer-readable media as used herein includes both storage media and communication media.

In further reference to FIG. 5, the computing system 500 may include global positioning system (“GPS”) circuitry 585 that can automatically discern its location based on relative positions to multiple GPS satellites. As described further below, GPS circuitry 585 may be used to determine a location of the computing system 500 particularly when computing system 500 is a mobile device like a smartphone.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While the disclosed subject matter has been described in terms of illustrative embodiments, it will be understood by those skilled in the art that various modifications can be made thereto without departing from the scope of the claimed subject matter as set forth in the claims.

What is claimed is:

1. A non-transitory computer readable medium having stored thereon software instructions that when executed by a processor, cause the processor to execute the method, comprising:
receiving from an administrator device a set of actions for a user to carry out;
receiving from the administrator device subtasks of the actions;
storing the actions and subtasks into a database;
receiving from the database, assessment factors, mood factors, and actions and tasks completed related to a user; and
sending to a user device, associated with the user, a subtask to be completed.

2. The non-transitory computer readable medium of claim 1, further comprising:
receiving information related to the subtask from the user device after the user has completed the subtask on the user device.

3. The non-transitory computer readable medium of claim 1, further comprising:
sending, to the user device, information related to actions and tasks completed.

4. The non-transitory computer readable medium of claim 1, further comprising:
sending, to the user device, information related to frequency of completing subtasks.

5. The non-transitory computer readable medium of claim 1, further comprising:
sending, to the user device, information related to history of completing subtasks.

6. The non-transitory computer readable medium of claim 1, wherein the processor is part of a computer server accessed over a communications network.

7. A method comprising:
starting a software application on a user device;
logging in to a user account;
providing user check-in information to the software application;
sending the check-in information to a server in communication with a database;
retrieving a daily task from the database; and
presenting the daily task to the user.

8. The method of claim 7, wherein the check-in information includes mood.

9. The method of claim 7, further comprising:
rejecting the task by the user.

10. The method of claim 9, further comprising:
providing a new task to the user.

11. The method of claim 7, further comprising:
retrieving habit dynamics information.

12. The method of claim 7, wherein the habit dynamics information includes at least one of day of the week, frequency of use, and notifications.

13. The method of claim 7, wherein the daily task includes journaling, list making, communicating with another, writing a letter, and imagining.

14. The method of claim 7, wherein the daily task includes group discussions.

15. The method of claim 7, wherein the daily task includes joining a community.

16. A method of content creation for a self-help software application, comprising:
extracting from a reference material at least one of exercises, contentions, and wisdom;
converting the at least one of exercises, contentions, and wisdom into actions;
associating a quote from the reference material with the at least one of the at least one of exercises, contentions, and wisdom;
breaking the actions down into one or more steps that are day-by-day tasks for completing an action; and
explaining the actions as instructions.

17. The method of claim 16, further comprising:
presenting a step to a user of the software application running on an electronic device.

18. The method of claim 17, wherein the electronic device includes a smartphone.

19. The method of claim 16, further comprising:
storing the instructions in a database.

20. The method of claim 16, further comprising:
storing the steps in a database.
